(19) United States
(12) Patent Application Publication
(10) Pub. No.: US 2007/0282117 A1
(43) Pub. Date: Dec. 6, 2007

(54) Method of preparing enhanced reactive vegetable oils

(76) Inventors: Trevor Newbold, Bentonville, AR (US); David M. Casper, Normal, IL (US)

Correspondence Address: MCKELLAR IP LAW, PLLC, 784 SOUTH POSEYVILLE ROAD, MIDLAND, MI, 48640, US

(21) Appl. No.: 11/445,420

(22) Filed: Jun. 1, 2006

(51) Int. Cl. C07C 67/26

(52) U.S. Cl. 554/149

(57) ABSTRACT

A method of preparing enhanced reactive vegetable oils wherein the method comprises providing a hydroxy functional vegetable oil having a predetermined hydroxyl value and, under nitrogen, treating the hydroxyl functional vegetable oil with a catalyst using heat and pressure. Any water that is formed is removed. The mixture is heated under pressure and then an alkylene oxide is added while heating under pressure. In one embodiment, thereafter, there is added ethylene oxide and the material is heated and then the material is neutralized with acid. This process results in a primary hydroxyl functional vegetable oil polyol that is an enhanced reactive vegetable oil.

### BACKGROUND OF THE INVENTION

Most recently, vegetable oils, after being converted to a hydroxy form, have been utilized as co-reactants in conjunction with isocyanates, in polyurethane foam systems in the place of synthetic polyols.

Commercially available vegetable oils have been in existence for a long time and just recently, commercially viable methods were devised to convert such vegetable oils from the triglyceride structures containing both saturated and unsaturated moieties, to the hydroxy variety.

Such hydroxy functional compounds can then be made useful, for example, in the formation of urethanes by reacting the hydroxy groups with isocyanates. Coatings, adhesives, elastomers, foams and composites can be made from elastomeric compositions using such hydroxy functional compounds For most polyurethane foam systems, those hydroxy functional vegetable oils can be used. However, in the flexible polyurethane market, that includes slab stock foam, new and novel polyols have to be used.

Polyols having secondary alcohols are preferred in the slab stock market for their reduced activity in the polyurethane reaction thereby reducing the possibility of scorching from heat buildup. The polyether portion of the chain allows for the spring-like flexibility and rebound required in such slab polyurethane foam.

Much more recently, methods have been developed for providing “pure” hydroxy functional vegetable oils, such as those described in co-pending U.S. patent application Ser. No. 10/924,332, filed on Aug. 23, 2004 in the name of the inventors herein, wherein polyols made from vegetable oils are provided by a process that produces “pure” hydroxy functional vegetable oils having properties such as freeze/thaw stability, low odor, color of less than 0.5, acid numbers of less than 1.0 mg KOH/g, no residual peroxygens, less than 0.01% w/w of water, less than 0.01% w/w of organic acids, using AOCS Official Process CD-22-91 wherein the results are reported as %(A/A) and using the following standard process of analysis: AOCS Official Process, DC 3d-63 for acid value; AOCS Official Process, Cd 1-25 for Iodine value of fats and oils, Wijs Process; AOCS Official Process, c 13-60 for hydroxyl values, and AOCS Official Process, Cc 13c-50 color spectrophotometer process. Low odor for this of this type of polyol means that the polyols have a rating of 7 or greater on the SAE J1351 test wherein the rating scale of the SAE J 1351 test is replaced by the GME 60276 rating scale. For purposes of this invention, “freeze/thaw stability” means at least 5 cycles of freeze/thaw.

The above-mentioned U.S. patent application is incorporated herein by reference for what it teaches about pure vegetable oils and methods for their preparation. It should be understood that this invention is based on the use of such “pure” vegetable oils, and it is believed by the inventors herein that such “pure” vegetable oils have not been used heretofore for this purpose and it was not therefore known that such “pure” vegetable oils could be converted into enhanced reactive vegetable oils without interfering in the structure or reactivity of such “pure” vegetable oils.

### BRIEF DESCRIPTION OF THE DRAWINGS

FIG. 1 is a schematic of the formula for a typical starting polyol for this invention.

FIG. 2 is a schematic of the formula for a propoxylated polyol intermediate of this invention.

FIG. 3 is a schematic of the formula for an ethoxylated polyol intermediate of this invention.

### THE INVENTION

The invention described and claimed herein deals with a method of preparing enhanced reactive vegetable oils wherein the method comprises providing a hydroxy functional vegetable oil having a predetermined hydroxyl value and, under nitrogen, treating the hydroxyl functional vegetable oil with a catalyst using heat and pressure. In a next step, any water that is formed is removed. The mixture is heated to at least 120° C. under pressure and then at least two equivalents of an alkylene oxide selected from the group consisting of ethylene oxide and propylene oxide, for each equivalent of hydroxyl in the starting materials are added while heating to 140° C. to 170° C. at no more than about 75 psig pressure for at least one hour to form an alkylene oxidated vegetable oil having terminal hydroxyl groups.

In another embodiment of this invention, the invention described and claimed herein deals with a method of preparing enhanced reactive vegetable oils having secondary hydroxyls and primary hydroxyls, wherein the method comprises providing a hydroxy functional vegetable oil having a predetermined hydroxyl value and, under nitrogen, treating the hydroxyl functional vegetable oil with a catalyst using heat and pressure.

In a next step, any water that is formed is removed. The mixture is heated to at least 120° C. under pressure and then at least two equivalents of an propylene oxide for each equivalent of hydroxyl in the starting materials are added while heating to 140° C. to 170° C. at no more than about 75 Psig pressure for at least one hour to form a propylene oxide terminated vegetable oil having terminal secondary hydroxyl groups.

The material is then cooled and there is added at least one equivalent of ethylene oxide for each equivalent of terminal secondary hydroxyl group that has been formed.

Thereafter, the material is heated to no greater than about 170° C. for at least thirty minutes and then the material is neutralized with acid for up to two hours at a temperature of up to about 120° C. This process results in a primary hydroxyl functional vegetable oil polyol.

As set forth Supra, the vegetable oils useful in this invention are pure vegetable oils. The average molecular weight of the pure vegetable oil starting material used in this invention ranges from about 1000 average molecular units to about 4000 average molecular units. Treatment by the inventive process described herein will render a primary alcohol functional vegetable oil having a molecular weight in the range of about 2500 average molecular units to about 8000 average molecular units.

Products having these molecular weight ranges are primarily targeted to the flexible polyurethane market, which includes slab stock, These new materials have to have such characteristics as low hydroxy value, high molecular weight and have a functionality of at least three hydroxyl groups. Propoxylation in this invention adds molecular weight to the hydroxylated vegetable oil and the ethoxylation adds primary alcoholic functionality to give an enhanced reactivity to the polyol for use in polyurethane foam preparation.

### DETAILED DESCRIPTION OF THE INVENTION

In preparing an enhanced reactive vegetable oil wherein the polyoxyalkylene chain contains a propylene oxide, an amount of propylene oxide calculated to provide the desired degree of propoxylation is introduced and the resulting mixture is allowed to react until the propylene oxide is consumed, as indicated by, for example, a drop in reaction pressure. Usually, the final product is treated with weak acid to neutralize any basic catalyst residues to provide the commercial product having the primary alcohols on the molecule.

In preparing an enhanced reactive vegetable oil wherein the polyoxyalkylene chain contains a ethylene oxide, an amount of ethylene oxide calculated to provide the desired degree of ethoxylation is introduced and the resulting mixture is allowed to react until the ethylene oxide is consumed, as indicated by, for example, a drop in reaction pressure. Usually, the final product is treated with weak acid to neutralize any basic catalyst residues to provide the commercial product having the primary alcohols on the molecule.

In preparing an enhanced reactive vegetable oil wherein the polyoxyalkylene chain contains a propylene oxide first block and an ethylene oxide second block, an amount of propylene oxide calculated to provide the desired degree of propoxylation is introduced and the resulting mixture is allowed to react until the propylene oxide is consumed, as indicated by, for example, a drop in reaction pressure. A similar introduction and reaction of a calculated amount of ethylene oxide serves to provide the second block that completes the reaction. Usually, the final product is treated with weak acid to neutralize any basic catalyst residues to provide the commercial product having the primary alcohols on the molecule.

It should be understood that each separate procedure serves to introduce a desired average number of alkylene oxide units per vegetable oil molecule. Thus, for example, the initial treatment of an hydroxylated vegetable oil mixture with q moles of propylene oxide per mole of hydroxylated vegetable oil serves to effect the propoxylation of each hydroxy group with propylene oxide to an average of m propylene oxide moieties per hydroxy group on the vegetable oil, although some hydroxy groups will have become combined with more than m propylene oxide moieties and some will have become combined with less than m. In general, the maximum number of propylene units in a single molecule will not exceed 8 and the number of ethylene units in a single molecule will not exceed 30. The variation in the number of alkylene oxide moieties is not critical as long as the average for the number of units in each block is within the limits set out above.

Each reaction is conducted at an elevated temperature and pressure. Suitable reaction temperatures are from about 120° C. to about 220° C., preferably, 130° C. to 180° C. and more preferably, 140° C. to 150° C. A suitable reaction pressure is achieved by introducing to the reaction vessel the required amount of propylene oxide or ethylene oxide, each of which has a high vapor pressure at the desired reaction temperature. The pressure serves as a measure of the degree of reaction and each reaction is considered to be complete when the pressure no longer decreases with time.

For best results, it is desirable to carry out the reactions under relatively moisture-free conditions and to avoid side reactions that form water. To dry the reaction vessel and connection, they may be swept out with dry, oxygen-free gas, for example nitrogen, before introducing the charge of alkylene oxides. The catalyst or catalyst mixtures should also be dry, or substantially dry. The propylene oxide and ethylene oxide should preferably be purified to remove moisture and any impurities that are capable of entering into side reactions that yield water.

Catalysts that are useful in this invention are alkali metal hydroxide, such as sodium hydroxide and potassium hydroxide, sodium ethoxide, sodium methoxides, alkali metal acetates, Lewis Acids, such as BF₃, and amines, such as trimethyl amine, or other tertiary amines, and mixtures thereof. Preferred catalysts are the alkali metal hydroxides and the sodium ethoxide and sodium methoxide and much preferred catalysts are sodium hydroxide and potassium hydroxide.

Catalysts used in this invention should be used in the range of from about 0.2 weight % to 1.0 weight %, and preferred is a use in the range of about 0.3 weight % to 0.75 weight %, the amount of catalyst being based on the total amount of the reactive components of the reaction. Typically in this invention the catalysts are added to the hydroxylated vegetable oil prior to the introduction of the alkylene oxides.

The instant process serves to provide high molecular weight enhanced reactive vegetable oils. What is meant by “high molecular weight” for purposes of this invention, is that the final products should have a molecular weight in excess of 2500 average molecular units and ranges up to about 8000 average molecular units.

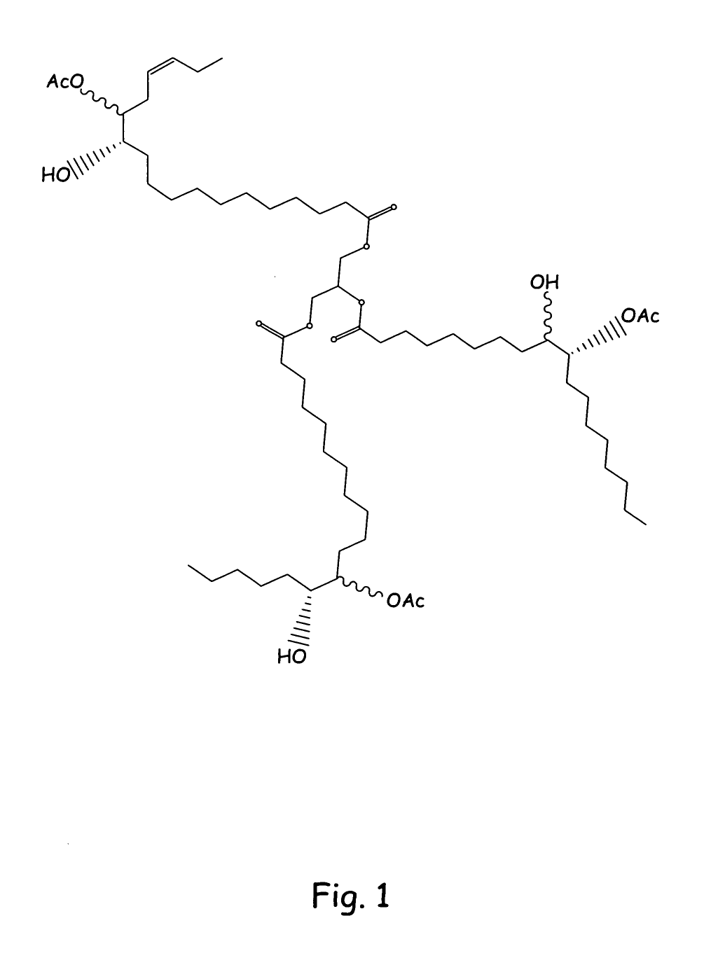

FIG. 1 shows a schematic of a formula for the starting material of this invention wherein the molecular weight of about 1100 average molecular units is shown.

### EXAMPLES

### Example 1

### Drying the Starting Material

A twenty gallon autoclave was charged with a total of 16,185 grams (13.9 mols) of a material of FIG. 1 and 80 grams of 45 weight % potassium hydroxide solution. An agitator located in the reactor was turned on and set to a speed of 75 rpm. A total of three pressure release cycles to 50 psig were performed with nitrogen and the reactor was heated to about 120° C. During the heat up, the nitrogen was sparged through the reaction mass to help remove water that was introduced with the catalytic KOH and the starting material. The reactor temperature was held at 120° C. for one hour with a nitrogen sparge. By the end of this hold time, the water content of the reaction mass was determined to be 80 ppm.

### Example 2

### Preparation of the Propylene Oxide Material

After the reaction mass in example 1 had been dried, the reactor temperature was increased to 155° C. and the agitator rate was increased to 300 rpm. Once this temperature had been achieved, the reactor pressure was increased to 10 psig with nitrogen, and one pound of propylene oxide was introduced to the reactor through a dip tube. The resulting pressure was 30 psig. After five minutes the reactor pressure started to drop and a mild exotherm was observed. At this point a continuous propylene oxide feed was started at a rate to keep the temperature between 150° C. and 160° C. and a pressure at or below 75 psig. A total of 33,056 grams (570 mols) of propylene oxide was fed to the reactor over a 4.5 hour period. This amount provides an average of 38.735 moles of propylene oxide per mole of the starting material. Each arm of the triglyceride has about 17.3 successively linked propylene oxide segments to form the polyether chain, terminating in a secondary alcohol.

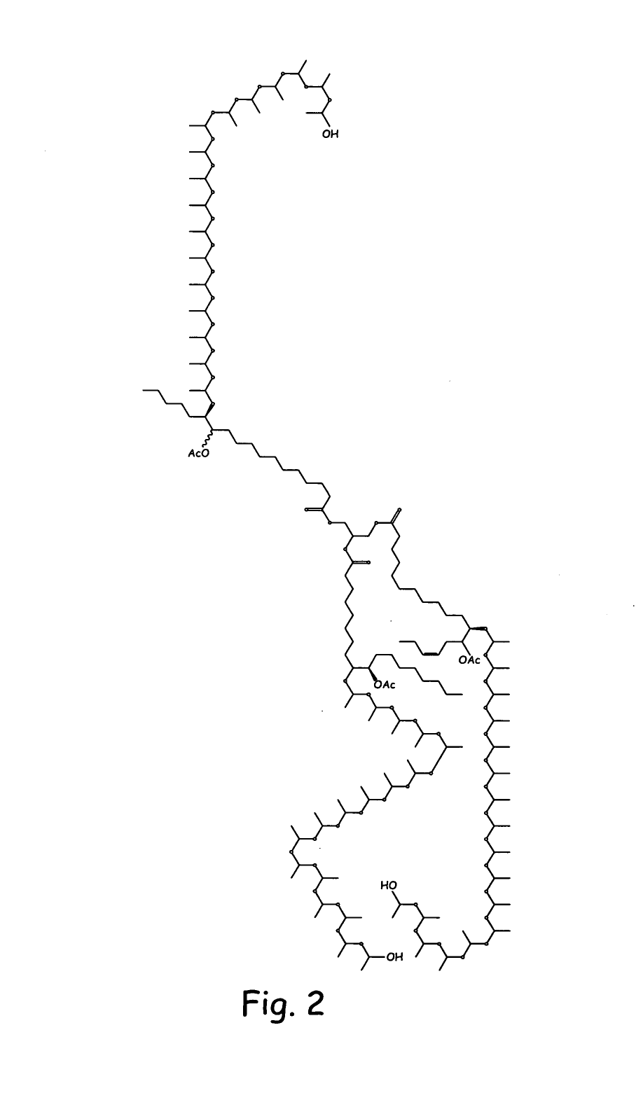

When the propylene oxide feed was complete, the reaction mass was held at 155° C. for one hour. During this hold period the reactor pressure dropped from just under 75 psig to near 10 psig. At this point, a sample was taken and the hydroxyl value of the sample was determined to be 50.8. This is the material shown schematically in FIG. 2.

### Example 3

### Preparation of the Propylene/Ethylene Oxide Product

At this time, the reactor was cooled to 60° C. and the reaction mass from example 2 was drained into five gallon pails. A total of 47,942 grams of product was recovered. Of this amount, 22,755 grams of product was returned to the reactor and heated to 155° C. after three pressure release cycles with nitrogen were performed. After heating the reactor was then pressured to 10 psig with nitrogen, the agitator was set to 300 rpm and 930 grams of ethylene oxide was charged through the dip tube. The resulting pressure was 60 psig. Over a fifteen minute period the pressure in the reactor had returned to 10 psig. The reaction mass was then held at 155° C. for an additional 30 minutes.

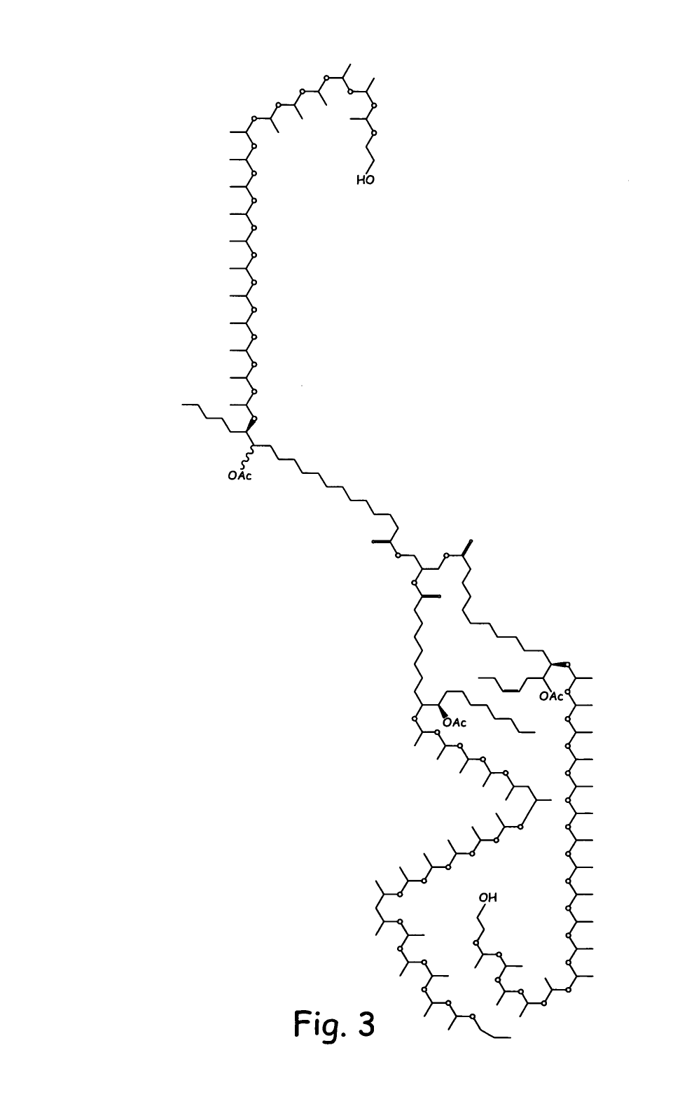

The agitator was slowed to 75 rpm and the reactor was cooled to 90° C. When the temperature reached 90° C., 50 grams of glacial acetic acid was introduced into the reactor through the dip tube. This was allowed to react for ten minutes. After this neutralization step, the reactor was again heated to 120° C. and a nitrogen sparge was started. The reactor was held at these conditions for one hour then cooled to 60° C. and then packaged. A total of 23,540 grams of the final product was recovered. This is the material shown schematically in FIG. 3. Ethylene oxide capping provides the polyether chain just as described above, but instead of the chain terminating with secondary alcohol, it terminates with a primary alcohol. Primary alcohols are more reactive than secondary alcohols, and provide the correct type of reactivity desired.

What is claimed is:

1. A method of preparing enhanced reactive vegetable oils, the method comprising:
(I) providing a pure hydroxy functional vegetable oil having a predetermined hydroxyl value;
(II) under, nitrogen, treating the pure hydroxyl functional vegetable oil with a catalyst using heat and pressure;
(III) removing any water formed in (II) from the material of (II).;
(IV) heating the mixture from (III) to at least 120° C. under pressure and, adding at least two equivalents of ethylene oxide for each equivalent of hydroxyl in (I) and heating to 140° C. to 170° C. at not more than about 75 psig pressure for at least one hour to form ethoxylated vegetable oils having terminal primary hydroxyl groups.

2. A method of preparing enhanced reactive vegetable oils, the method comprising:
(I) providing a pure hydroxy functional vegetable oil having a predetermined hydroxyl value;
(II) under, nitrogen, treating the pure hydroxyl functional vegetable oil with a catalyst using heat and pressure;
(III) removing any water formed in (II) from the material of (II).;
(IV) heating the mixture from (III) to at least 120° C. under pressure and, adding at least two equivalents of propylene oxide for each equivalent of hydroxyl in (I) and heating to 140° C. to 170° C. at not more than about 75 psig pressure for at least one hour to form propoxylated vegetable oils having terminal secondary hydroxyl groups.

3. A method of preparing enhanced reactive vegetable oils, the method comprising:
(I) providing a pure hydroxy functional vegetable oil having a predetermined hydroxyl value;
(II) under, nitrogen, treating the pure hydroxyl functional vegetable oil with a catalyst using heat and pressure;
(III) removing any water formed in (II) from the material of (II);
(IV) heating the mixture from (HI) to at least 120° C. under pressure and, adding at least two equivalents of propylene oxide for each equivalent of hydroxyl in (I) and heating to 140° C. to 170° C. at not more than about 75 psig pressure for at least one hour to form propoxylated vegetable oils having terminal secondary hydroxyl groups;
(V) cooling the material from (IV) and adding at least one equivalent of ethylene oxide for each equivalent of terminal secondary hydroxyl group in (V);
(VI) thereafter heating the material of (V) to no greater than about 170° C. for at least thirty minutes and then neutralizing the material of (VI) with acid for up to two hours at a temperature of up to about 120° C.
